(19) United States
(12) Patent Application Publication
(10) Pub. No.: US 2004/0176471 A1
(43) Pub. Date: Sep. 9, 2004

(54) Novel chalcone derivatives and pharmaceutical compositions comprising the same

(75) Inventors: Chun-Nan Lin, Kaohsiung (TW); Jih-Pyang Wang, Taichung (TW)

Correspondence Address: Paul D. Greeley, Esq., Ohlandt, Greeley, Ruggiero & Perle, L.L.P., One Landmark Square, 10th Floor, Stamford, CT, 06901-2682, US

(73) Assignee: KAOHSIUNG MEDICAL UNIVERSITY

(21) Appl. No.: 10/383,329

(22) Filed: Mar. 7, 2003

(51) Int. Cl. A61K 31/12; C07C 49/84

(52) U.S. Cl. 514/685; 568/334

(57) ABSTRACT

Disclosed herein are novel chalcone derivatives of formulas (I), (II) and (III):

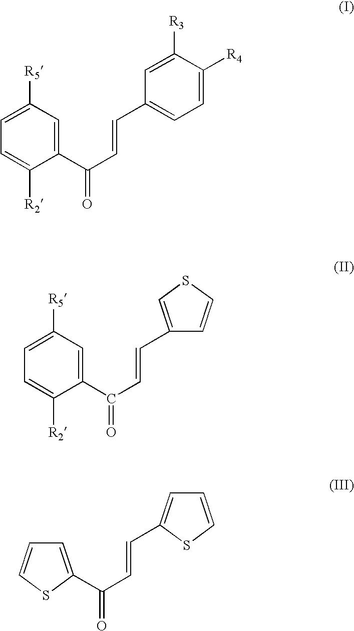

wherein each of the substituents is given the definition as set forth in the Specification and Claims. These compounds are demonstrated to have anti-inflammatory activities and thus can be used in the treatment of an inflammatory disorder in a subject.

### BACKGROUND OF THE INVENTION

1. Field of the Invention

This invention relates to novel chalcone derivatives, which are found to have anti-inflammatory activities, and their uses in the manufacture of pharmaceutical compositions.

2. Description of the Related Art

It is reported in literature that mast cells play a central role in the pathogenesis of diseases such as allergic asthma, rhinoconjunctivitis, urticaria, anaphylaxis and systemic mastocytosis, and might be important in other chronic inflammatory disorders (Middleton Jr. E., Kandaswami, C. (1992), Biochem. Pharmacol., 43: 1167-1179). In addition, it is known from literature that neutrophil is an important inflammatory cell and, upon being triggered by a variety of inflammatory stimuli, it can produce highly reactive oxygen species which have potent microbiocidal and inflammatory effects (Babior, B. M. (1978), N. Engl. J. Med., 298: 659-668; A. I. Tauber and B. M. Babior (1985), Free Radic. Biol. Med., 1: 265-307). Hence, inhibition of the activation of these inflammatory cells appears to be an important therapeutic target for small molecule drug design for the treatment of inflammatory diseases.

It is also known that macrophages are important In nonspecific host resistance to microbial pathogens and serve as central regulators, of the specific immune response (W. Solbach et al. (1991), Immunol. Today, 12: 4-6). Upon activation, nitric oxide (NO), together with other chemical mediators, will be released from macrophages in response to bacterial endotoxin (lipopolysaccharide, LPS) (A. H. Ding et al. (1988), J. Immunol., 141: 2407-2412).

It is further known that microglial cells, the resident macrophages in brain, have detrimental as well as beneficial effects on the surrounding cells and are believed to be involved in most inflammatory, infectious and degenerative diseases of the central nerve system (CNS) (M. Mattat and B. Chamak (1994), J. Leukoc. Biol. 56: 416-422; J. Gehrmann et al. (1995), Brain Res. Rev., 20: 269-287). The release of NO by activated microglial cells in response to LPS is likely to have a crucial role in mediating the interaction between microglia and other cells present in the nervous system, as it is known to regulate inflammation, immune functions, blood vessel dilation, neurotransmission and neural cell survival (P. J. Gebicke-Haerter et al. (1989), J. Neurosci., 9: 183-194; C. Nathan (1992), FASEB J. 6: 3051-3064; L. Minghetti et al. (1997), Glia 19: 152-160). Hence, inhibition of NO released from microglial cells or macrophages is a rational therapeutic approach to treat a variety of inflammatory and neuronal conditions observed in aging and Alzheimer's disease (L. Meda et al. (1995), Nature, 374: 647-650).

3,4-Dihydroxychalcones have been reported as 5- or 12-lipoxygenase and cyclooxygenase inhibitors (Y. Sogawa et al. (1993), J. Med. Chem., 36: 3904-3909). Some chalcone derivatives have been reported to exert potent anti-inflammatory effects, at least partly, through the suppression of chemical mediators released from mast cells, neutrophils, macrophages, and microglial cells in vitro, and to suppress the edematous response in vivo (C. N. Lin et al. (1997), J. Pharm. Pharmacol., 49: 530-536; H. K. Hsieh et al. (1998), Pharmceu. Res., 15 (1): 39-46; H. K. Hsieh et al. (2000), J. Pharm. Pharmacol., 52: 163-171). These findings suggest that some chalcones might be promising anti-inflammatory agents.

Brousschalcone A, which is a natural chalcone product isolated from the cortex of Broussonetia papyrifera (Moraceae) and which has the following chemical structure, has a potent antiplatelet effect and is a potent inhibitor of cyclooxygenase. At a concentration of 3 mg/mL, it has a significant inhibitory effect on the release of β-glucuronidase (% inhibition, 51.1±12.6) and lysozyme (% inhibition, 68.0±13.4) from rat neutrophils stimulated with formyl-Met-Leu-Phe (fMLP). In addition, Brousschalcone A exerts a potent antioxidant activity and inhibits the respiratory burst in neutrophils and the inducible nitric oxide synthase (iNOS) expression in macrophages (C. N. Lin et al. (1996), J. Pharm. Pharmacol., 48:532-538; J.-P. Wang et al. (1997), Eur. J. Pharmacol 320, 201; Z.-J. Cheng et al. (2001), Biochem. Pharmacol., 61, 939).

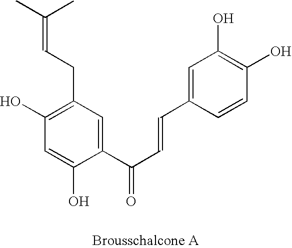

NO plays a central role in macrophage-induced cytotoxicity and excess NO may contribute to the pathophysiology of septic shock (C. Thiermermann and J. R. Vane (1990), Eur. J. Pharmacol., 182, 591-595). Some 2′,5′-Dialkoxychalcones and 2′,5′-dihydroxy-4-chloro-dihydrochalcone have been reported to inhibit nitric oxide (NO) production in lipopolysaccharide (LPS)/interferon-γ (IFN-γ)-activated N9 microglial cells and in LPS-activated RAW 264.7 macrophage-like cells. These compounds also suppressed the inducible NO synthase (iNOS) expression and cyclooxygenase-2 (COX-2) activity in RAW 264.7 cells (Hsin-Kaw. Hsieh et al. (2000), J. Pharm. Pharmacol., 52, 163-171). These findings suggested that some chalcones may be promising anti-inflammatory agents and have potential in the therapy of septic shock.

In an effort to develop new potent anti-inflammatory agents, the Applicant has synthesized a series of new chalcone derivatives and evaluated their inhibitory effects on the activation of mast cells, neutrophils, macrophages, and microglial cells

### SUMMARY OF THE INVENTION

Accordingly, in the first aspect, the present invention provides a compound of formula (I):

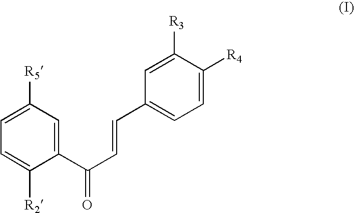

wherein

R₂′ and R₅′ are the same and represent OR, where R is H or a C₁₋₆ alkyl group; or R₂′ is OH and R₅′ is propoxy;

R₃ represents H or Cl; and

R₄ represents OH, Cl or Br;

with the proviso that when R₃ is H, R₄ cannot be Cl.

In the second aspect, the present invention provides a compound of formula (II) or a pharmaceutically acceptable salt thereof:

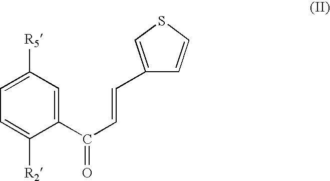

wherein both R₂′ and R₅′ are OH

In the third aspect, the present invention provides a compound of formula (III) or a pharmaceutically acceptable salt thereof:

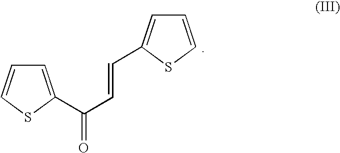

The above-described compounds of formulas (I), (II) and (III) are found to have anti-inflammatory effects. Therefore, in the fourth aspect, the present invention provides a pharmaceutical composition comprising as an active ingredient a compound of formula (I) or (II) or (III), in its free form or a pharmaceutically acceptable salt thereof.

In the fifth aspect, the present invention provides a method for treating an inflammatory disorder in a subject, comprising administering to a subject in need of such treatment a therapeutically effective amount of a compound of formula (I) or (II) or (III), in its free form or a pharmaceutically acceptable salt thereof.

The above and other objects, features and advantages of the present invention will become apparent with reference to the following detailed description of the preferred examples.

### DETAILED DESCRIPTION OF THE INVENTION

In this invention, the Applicant synthesized new chalcone derivatives via Claisen-Schmidt condensation reaction using appropriate acetophenones or hydroxyacetophenones, protected as tetrahydropyranyl ether, with appropriate aromatic aldehydes or hydroxyaromatic aldehydes, protected as tetrahydropyranyl ether. These new chalcone derivatives may be synthesized according to the following general synthesis scheme:

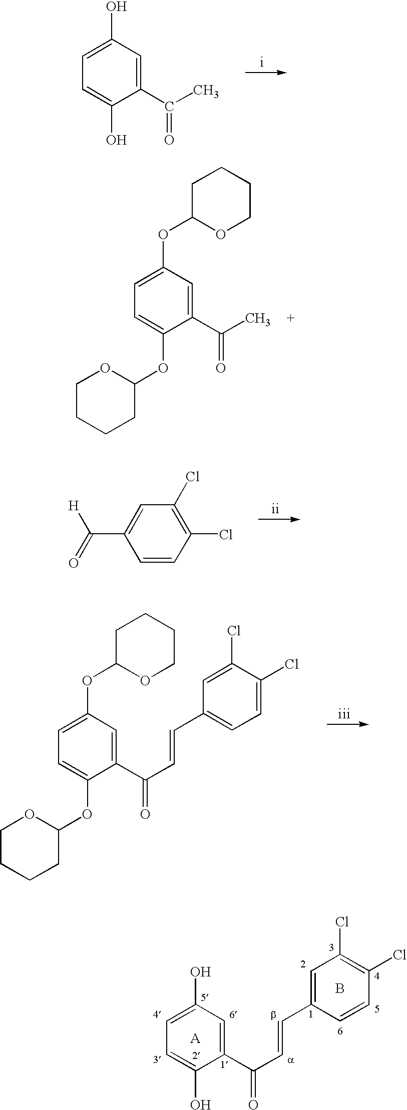

Reagents: (i) pyridinium p-toluenesulfonate, 3,4-dihydro-α-pyran, room temperature, 4 hrs:

(ii) BaOH.8H₂O, 40° C., HCl;

(iii) p-toluenesulfonic acid, room temperature, 4 hrs, 5% NaHCO₃.

The resultant 3,4dichloro-2′,5′-dihydroxychalcone from step (iii) may be further subjected to an alkylation reaction in the presence of an appropriate C₁₋₆ alkyl halide, K₂CO₃ and DMF at room temperature, to thereby result in a corresponding 3,4-dichloro-2′,5′-dialkoxychalcone or 3,4-dichloro-2′-hydroxy-5′-alkoxychalcone.

Therefore, this invention provides a compound of formula (I):

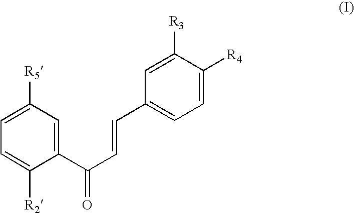

wherein

R₂′ and R₅′ are the same and represent OR, where R is H or a C₁₋₆ alkyl group; or R₂′ is OH and R₅′ is propoxy;

R₃ represents H or Cl; and

R₄ represents OH, Cl or Br;

with the proviso that when R₃ is H, R₄ cannot be Cl.

Preferably, both R₃ and R₄ are Cl. In a preferred embodiment of this invention, both R₂′ and R₅′ are OH. In another preferred embodiment of this invention, both R₂′ and R₅′ are ethoxy. In a further preferred embodiment of this invention, R₂′ is OH and R₅′ is propoxy.

Preferably, both R₂′ and R₅′ are methoxy. In a preferred embodiment of this invention, R₃ is H and R₄ is OH. In another preferred embodiment of this invention, R₃ is H and R₄ is Br. In a further preferred embodiment of this invention, both R₃ and R₄ are Cl.

This invention also provides a compound of formula (II):

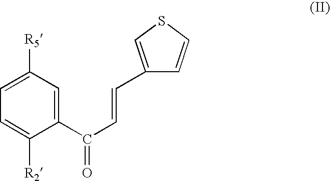

wherein both R₂′ and R₅′ are OH.

This invention further provide a compound of formula (III):

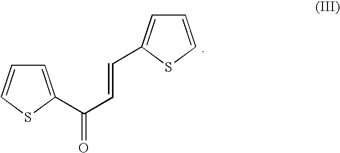

According to this invention, the compounds of formulas (I), (II) and (III) may be in their free form or in the form of a pharmaceutically acceptable salt thereof.

Illustrative pharmaceutically acceptable salts include metal salts such as sodium salt, potassium salt, calcium salt, magnesium salt, manganese salt, iron salt and aluminum salt; mineral acid addition salts such as hydrochloride, hydrobromide, hydroiodide, sulfate and phosphate; organic acid addition salts such as benzoate, methanesulfonate, ethanesulfonate, benzenesulfonate, p-toluenesulfonate, oxalate, maleate, fumarate, tartrate and citrate; and those with amino acids, such as arginine, aspartic acid and glutamic acid.

In addition, the compound of formula (I), (II) or (iii) of the present invention may also exist in the form of solvates represented by the hydrate. Such solvates should also be included in the present invention.

The compounds according to this invention have been demonstrated to have anti-inflammatory effects, including inhibiting the release of β-glucuronidase and lysozyme from rat neutrophils stimulated with formyl-Met-Leu-Phe (fMLP)/cytochalasin B (CB), inhibiting superoxide anion generation in rat neutrophils in response to tMLP/CB, and inhibiting NO production in macrophages and microglial cells. It is therefore contemplated that the compounds of this invention can be used in the manufacture of pharmaceutical compositions for use in the treatment of an inflammatory disorder in a subject.

The inflammatory disorder may be one associated with the release of chemical mediators from inflammatory cells selected from macrophages, neutrophils and microglial cells, or one associated with NO production by inflammatory cells selected from macrophages, neutrophils and microglial cells. In particular, the inflammatory disorder is selected from pathologies of central neurologic diseases, peripheral tissue damages associated with acute or chronic inflammation, inflammatory/neuronal conditions observed in aging and Alzheimer's disease, and septic chock.

The unit dosage form of the pharmaceutical compositions may, in accordance with the object of a therapy, be suitably chosen from any one of oral preparations, injections, suppositories, ointments, inhalants, eye drops, nasal drops, plasters and the like. These unit dosage forms can each be prepared by a preparation method commonly known and used by those skilled in the art.

To produce an oral solid preparation, an excipient and, if necessary, a binder, a disintegrator, a lubricant, a coloring matter, a flavoring agent and/or the like may be admixed with a compound of the present invention. The resultant mixture can then be formed into tablets, coated tablets, granules, powder, capsules or the like by a method known per se in the art. Such additives can be those generally employed in the present field of art, including excipients, lactose, sucrose, sodium chloride, glucose, starch, calcium carbonate, kaolin, micro-crystalline cellulose, and silicic acid; binders: water, ethanol, propanol, sucrose solution, glucose solution, starch solution, gelatin solution, carboxymethylcellulose, hydroxypropyl-cellulose, hydroxypropylstarch, methylcellulose, ethylcellulose, shellac, calcium phosphate, and polyvinylpyrrolidone; disintegrators: dry starch, sodium alginate, powdered agar, sodium hydrogencarbonate, calcium carbonate, sodium lauryl sulfate, monoglycerol stearate, and lactose; lubricants: purified talc, stearate salts, borax, and polyethylene glycol; and corrigents: sucrose, bitter orange peel, citric acid, and tartaric acid.

To produce an oral liquid preparation, a flavoring agent, a buffer, a stabilizer and the like may be admixed with a compound of the present invention. The resultant mixture can then be formed into a solution for internal use, a syrup, an elixir or the like by a method known per se in the art. In this case, the flavoring agent can be the same as that mentioned above. Illustrative of the buffer is sodium citrate, while illustrative of the stabilizer are tragacanth, gum arabic, and gelatin.

To prepare an injection, a pH regulator, a buffer, a stabilizer, an isotonicity and the like may be admixed with a compound of the present invention. The resultant mixture can then be formed into a subcutaneous, intramuscular or intravenous injection by a method known per se in the art. Examples of the pH regulator and buffer include sodium citrate, sodium acetate, and sodium phosphate. Illustrative of the stabilizer include sodium pyrosulfite, EDTA, thioglycollic acid, and thiolactic acid. Examples of the isotonicity include sodium chloride and glucose.

To prepare suppositories, a pharmaceutical carrier known in the present field of art, for example, polyethylene glycol, lanolin, cacao butter or fatty acid triglyceride may be added, optionally together with a surfactant such as “Tween” (registered trademark), to a compound of the present invention. The resultant mixture can then be formed into suppositories by a method known per se in the art.

To prepare an ointment, a pharmaceutical base, a stabilizer, a humectant, a preservative and the like are combined, as needed, with a compound of the present invention. The resultant mixture can then be mixed and prepared into an ointment by a method known per se in the art. Illustrative of the pharmaceutical base are liquid paraffin, white petrolatum, white beewax, octyldodecyl alcohol, and paraffin. Examples of the preservative include methyl parahydroxybenzoate, ethyl parahydroxybenzoate, and propyl parahydroxybenzoate.

In addition to the above-described preparations, the compound of the present invention may also be formed into an inhalant, an eye drop and a nasal drop by methods known per se in the art.

The dosage of the pharmaceutical composition according to the present invention varies depending on the age, body weight, conditions, unit dosage form, administration frequency and the like. In general, however, it is preferred to orally or parenterally administer to a subject a compound of this invention as an effective ingredient in an amount of about 1 to 1,000 mg per day in one or several dosages.

### EXAMPLES

The present invention will be described in more detail with reference to the following examples, which are given for the purpose of illustration only and are not intended to limit the scope of the present invention.

General Procedures:

Melting points (uncorrected) were determined with a Yanaco Micro-Melting Point apparatus. IR spectra were determined with a Perkin Elmer system 2000 FT-IR spectrophotometer. ¹H (400 MHz) and ¹³C (100 MHz) NMR spectra were recorded on a Varian Unity-400 spectrometer, and Mass were obtained on a JMS-HX 100 mass spectrometer. Elemental analyses were within ±0.4% of the theoretical values, unless otherwise noted. Chromatography was performed using a flash-column technique on silica gel 60 supplied by E. Merck.

The starting compounds and reagents used in the following synthesis examples are commercially available from T.C.I. or Merck.

### Synthesis Example 1

### 2′,5′-Dimethoxy-4-hydroxychalcone (Compound 1)

1A Synthesis of 4-(tetrahydropyran-2-yloxy)benzaldehyde (Compound 1a)

4-Hydroxybenzaldehyde (3.05 g, 0.25 mmol) and pyridinium p-toluenesulfonate (0.15 g, 0.6 mmol) were stirred in methylene chloride (80 mL) for 0.5 hr and 3,4-dihydro-α-pyran in methylene chloride (13 mL in 20 mL) was then added dropwise. The resultant reaction mixture was stirred at room temperature for 4 hrs, washed twice with water, dried, and evaporated in vacuo. The obtained residue yielded crude 4-(tetrahydropyran-2-yloxy) benzaldehyde (compound 1a), and a part of the crude compound 1a was eluted through a silica-gel column with n-hexane/CH₂Cl₂ (2:1) to give a yellowish oil.

Detected Properties of the Title Compound:

¹H NMR (CDCl₃): δ 1.37, 1.66 (each 3H, m,

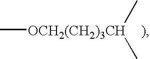

3.36 (1H, m, —OCHHCH₂—), 3.57 (1H, m, —CHHCH₂—), 5.27 (1H, t, J=2.8 Hz,

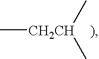

6.90 (2H, dd, J=8.8, 2.0 Hz, H-2 and H-6), 7.56 (2H, dd, J=8.8, 2.0 Hz, H-3 and H-5), 9.60 (1 H, s, —CHO).

¹³C NMR (CDCl₃): δ 17.7 (—CH₂CH₂CH₂—), 24.3 (—OCH₂CH₂—), 29.3 (

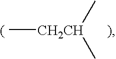

61.2 (—OCH₂—), 95.4

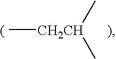

116.0 (C-2 and C-6), 129.7 (C-4), 131.0 (C-3 and C-5), 161.5 (C-1), 190.1 (—CHO).

EIMS (70 ev) m/z (% rel. int.): 206 (8) [M]⁺, 189 (10), 121 (29), 85 (100).

HREIMS m/z [M]′ 206.0941 (calcd. for C₁₂H₁₄O₃, 206.0943).

1B. Synthesis of 2′,5′-dimethoxy-4-(tetrahydropyran-2-yloxy) Chalcone (Compound 1b)

Crude compound 1a, 2,5-dimethoxyacetophenone (4.5 g, 25 mmol), and barium hydroxide octahydrate (4.29 g, 25 mmol) were dissolved in MeOH (100 mL) The resultant reaction mixture was stirred at 40° C. for 12 hrs and then evaporated in vacuo. Subsequent to the addition of water (100 mL), the resultant mixture was neutralized with HCl (1 M, 35 mL) and extracted with EtOAc. The organic layer was separated, washed with water, dried, and evaporated in vacuo. The obtained residue yielded crude 2′, 5′-dimethoxy-4-(tetrahydropyran-2-yloxy) chalcone (compound 1b), and a part of the crude compound 1b was eluted through a silica-gel column with n-hexane/CH₂Cl₂ (2:1) to give a yellowish oil.

Detected Properties of the Title Compound:

¹H NMR (CDCl₃): δ 1.56, 1.82 (each 3H, m,

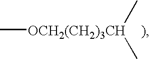

3.56 (1 H, m, —OCHCHCH₂—), 3.72 (3H, s, OMe), 3.78 (3H, s, OMe), 3.8 (1H, m, —OCHHCH₂—), 5.41 (1H, t, J=2.8 Hz,

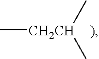

6.86 (1 H, d, J=8.8 Hz, H-3′), 6.91 (1H, dd, J=8.8, 2.8 Hz, H-4′), 7.02 (2H, dd, J=8.8, 2.8 Hz, H-3 and H-6), 7.14 (1H, d, J=2.8 Hz, H-6′), 7.27 (1H, d, J=15.8 Hz, H-α), 7.48 (2H, dd, J=8.8, 2.8 Hz, H-2 and H-6), 7.58 (1H, d, J=15.8 Hz, H-β).

¹³C NMR (CDCl₃): δ 18.3 (—CH₂CH₂CH₂—), 24.8 (—OCH₂C₂—), 29.8

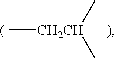

55.4 (OMe), 56.1 (OMe), 61.7 (—OCH₂—), 95.8

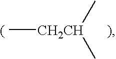

113.1 (C-6′), 114.2 (C-3′), 116.4 (C-3 and C-5), 118.4 (C-α), 124.6 (C-4′), 128.1 (C-1′), 129.7 (C-2 and C-6), 130.1 (C-1), 143.2 (C-β), 152.1 (C-5′), 153.2 (C-2′), 158.7 (C-4), 192.3 (CO).

EIMS (70 eV) m/z (% rel. int.): 368 (1) [M]³⁰ , 284 (100), 253 (21), 178 (86), 151 (48), 107 (57).

HREIMS m/z [M]⁺ 368.1630 (calcd. for C₂₂H₂₄O₅, 368.1623).

1C. Synthesis of 2′,5′-dimethoxy-4-hydroxychalcone (Compound 1).

Crude compound 1b and p-toluenesulphonic acid (0.18 g, 1.05 mmol) were dissolved in MeOH (100 mL). The reaction mixture was stirred at room temperature for 4 hrs, and then evaporated in vacuo. Water (100 mL) was added to the mixture, and the resultant mixture was neutralized with 5% NaHCO₃ (50 mL), followed by extraction with EtOAc. The organic layer was separated, washed with water, dried, and evaporated in vacuo. The residue was eluted through a silica-gel column with cyclohexane/EtOAc (4:1) to give the title compound 1 (3.41 g, 12 mmol, yield 48%).

Detected Properties of the Title Compound:

IR (KBr) 3319, 1650, 1558 cm⁻¹.

¹H NMR (CDCl₃): δ 3.79 (3H, s, OMe), 3.83 (3H, s, OMe), 6.88 (2H, dd, J=8.8, 2.0 Hz, H-3 and H-5), 6.93 (1H, d, J=8.8 Hz, H-3′), 7.02 (1H, dd, J=8.8, 2.8 Hz, H-4′), 7.15 (1H, d, J=2.8 Hz, H-6′), 7.25 (1H, d, J=16.0 Hz, H-α), 7.46 (2H, dd, J=8.8, 2.0 Hz, H-2 and H-6), 7.59 (1H, d, J=16.0 Hz, H-β).

¹³C NMR (CDCl₃): δ 55.8 (OMe), 56.5 (OMe), 113.5 (C-6′), 114.5 (C-3′), 116.1 (C-3 and C-5), 118.9 (C-α), 124.3 (C-4′), 127.3 (C-1′), 129.8 (C-1), 130.5 (C-2 and C-6), 144.6 (C-β), 152.4 (C-5′), 153.6 (C-2′), 158.6 (C-4), 193.5 (CO).

EIMS (70 eV) m/z (% rel. int.): 284 [M]⁺ (100), 177 (77), 147 (88), 107 (84).

### Synthesis Example 2

### 4-Bromo-2′,5′-dimethoxychalcone (Compound 2)

2,5-Dimethoxyacetophenone (4.5 g, 25 mmol), 4-bromobenzaldehyde (4.63 g, 25 mmol) and barium hydroxide octahydrate (4.29 g, 25 mmol) were treated according to the procedures as set forth in the above Synthesis Examples 1B for compound 1b to result in the title compound 2 (3.91 g, 11.3 mmol, yield 45%).

Detected Properties of the Title Compound:

IR (KBr) 1660, 1600 cm⁻¹.

¹H NMR (CDCl₃): δ 3.81 (3H, s, OMe), 3.86 (3H, s, OMe), 6.94 (1H, d, J=9.0 Hz, H-3′), 7.04 (1H, dd, J=9.0, 3.1 Hz, H-4), 7.19 (1H, d, J=3.1 Hz, H-6′), 7.41 (1H, d, J=15.8 Hz, H-α), 7.42-7.52 (4H, m, H-2, H-3, H-5 and H-6), 7.58 (1H, d, J=15.8 Hz, H-β).

¹³C NMR (CDCl₃) δ 55.8 (OMe), 56.4 (OMe), 113.4 (C-6′), 114.4 (C-3′), 119.4 (C-α), 124.3 (C-1′), 127.3 (C-4′), 129.1 (C-1), 129.7 (C-3, C-5), 132.0 (C-2, C-6), 134.1 (C-4), 141.5 (C-β, 152.6 (C-5′), 153.6 (C-2′), 191.9 (CO).

EIMS (70 eV) m/z (% rel. int.): 348 [M+1]⁺ (11), 177 (20),165 (100).

### Synthesis Example 3

### 3,4Dichloro-2′,5′-dimethoxychalcone (Compound 3)

2,5-Dimethoxyacetophenone (4.5 g, 25 mmol), 3,4-dichlorobenzaldehyde (4.38 g, 25 mmol) and barium hydroxide octahydrate (4.29 g, 25 mmol) were treated according to the procedures as set forth in the above Synthesis Examples 1B for compound 1b to result in the title compound 3 (4.05 g, 12 mmol, yield 48%).

Detected Properties of the Title Compound:

IR (KBr) 1669, 1605 cm⁻¹.

¹H NMR (CDCl₃): δ 3.81 (3H, s, OMe), 3.88 (3H, s, OMe), 6.95 (1H, d, J=9.2 Hz, H-3′), 7.05 (1H, dd, J=9.2, 3.2 Hz, H-4′), 7.20 (1H, d, J=32 Hz, H-6′), 7.41 (1H, d, J=16.0 Hz, H-α), 7.41 (1H, dd, J=8.4, 2.0 Hz, H-6), 7.47 (1H, d, J=8.4 Hz, H-5), 7.54 (1H, d, J=16.0 Hz, H-β) 7.66 (1H, d, J=2.0 Hz, H-2).

¹³C NMR (CDCl₃): δ 55.9 (OMe), 56.2 (OMe), 113.6 (C-6′), 114.5 (C-3′), 119.8 (C-α), 127.3 (C-4′), 128.4 (C-6), 129.2 (C-1′), 129.8 (C-2), 130.8 (C-5), 133.2 (C-1), 134.0 (C-3), 135.4 (C-4), 139.9 (C-β), 152.8 (C-5′), 153.7 (C-2′), 191.6 (CO).

EIMS (70 eV) m/z (% rel. int.): 336 [M−1]⁺ (11), 165 (100).

### Synthesis Example 4

### 3,4-Dichloro-2′,5′-dihydroxychalcone (Compound 4)

4A. Synthesis of 2′,5′-bis(tetrahydropyran-2-yloxy)acetophenone (Compound 4a)

2,5-Dihydroxyacetophenone (3.8 g, 25 mmol) and pyridinium P-toluenesulfonate (0.15 g, 0.6 mmol) were stirred in methylene chloride (80 mL) for 0.5 hr, and 3,4-dihydro-α-pyran in methylene chloride (13 mL in 20 mL) was then added dropwise. The resultant reaction mixture was stirred at room temperature for 4 hrs. Thereafter, the reaction mixture was washed twice with water, dried, and evaporated in vacuo. The obtained residue yielded crude 2′,5′-bis(tetrahydropyran-2-yloxy)acetophenone (compound 4a), and a part of the crude compound 4a was eluted through a silica-gel column with n-hexane/CH₂Cl₂ (2:1) to give a product as a yellowish oil, which was identified with various spectral data and compared with those of authentic sample (i.e. compound (3a) reported in H. K. Hsieh et at. (1998), Pharm. Res., 15, 39-46).

4B. Synthesis of 2′,5′-bis(tetrahydropyran-2-yloxy)-3,4-dichlorochalcone (Compound 4b)

Crude compound 4a (8.1 g, 25 mmol), 3,4-dichloro-benzaldehyde (4.4 g, 25 mmol) and barium hydroxide octahydrate (4.2 g, 25 mmol) were dissolved in MeOH (100 mL). The resultant reaction mixture was stirred at 40° C. for 12 h, and then evaporated in vacuo. Subsequent to the addition of water (100 mL), the resultant mixture was neutralized with HCl (1M, 30 mL) and extracted with EtOAc. The organic layer was separated, washed with water, dried, and evaporated in vacuo. The obtained residue yielded crude 2′,5′-bis(tetrahydropyran-2-yloxy)-3,4-dichlorochalcone (compound 4b), and a part of the crude compound 4b was eluted through a silica-gel column with n-hexane/CH₂Cl₂ (2:1) to give a product as a yellowish powder.

Detected Properties of the Title Compound:

¹H NMR (CDCl₃): δ 1.65 (6H, m,

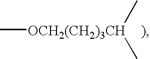

1.95 (6H, m,

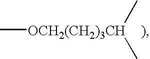

3.63 (2H, m, —OCH₂—), 3.94 (2H, m, —OCH₂—), 5.34 (2H, t, J=2.8 Hz,

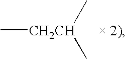

6.94 (1H, d, J=8.8 Hz, H-3′), 7.09 (1H, d, J=2.8 Hz, H-6′), 7.29 (1H, dd, J=8.8, 2.8 Hz, H-4′), 7.44 (1 H, d, J=9.0 Hz, H-3), 7.51 (1H, d, J=15.8 Hz, H-α), 7.53 (1H, dd, J=9.0, 3.0 Hz, H-2), 7.53 (1H, d, J=3.0 Hz, H-6), 7.75 (1H, d, J=15.8, H-β).

¹³C NMR (CDCl₃): δ 18.7 (—CH₂CH₂CH₂—), 19.7 (—CH₂CH₂CH₂—), 25.1 (—OCH₂CH₂—), 25.4 (—OCH₂CH₂—), 30.4 (

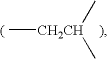

30.6

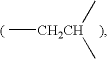

62.1 (—OCH₂—), 62.9 (—OCH₂—), 94.6 (

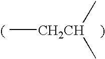

97.6

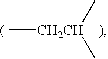

116.5 (C-3′), 119.2 (C-α), 119.4 (C-6′), 121.7 (C-4′), 127.6 (C-6), 130.0 (C-2), 130.9 (C-5), 133.3 (C-1), 134.0 (C-3), 134.7 (C-4), 142.5 (C-β), 149.1 (C-5′), 158.6 (C-2′), 192.7 (CO).

EIMS (70 eV) m/z (% rel. int.) 480 (0.02) [M+4]⁺, 478 (0.1), [M+2]⁺, 476 (0.2) [M]⁺, 394 (0.2), 392 (0.3), 310 (43), 308 (70), 136 (100).

Compound 4b failed to show a molecular ion peak under high-resolution conditions.

4C. Synthesis of 3,4-Dichloro-2′,5′-dihydroxychalcone (Compound 4)

Crude compound 4b (0.50 g, 1.05 mmol) and p-toluenesulfonic acid (0.18 g, 1.05 mmol) were-dissolved in MeOH (100 mL). The resultant reaction mixture was stirred at room temperature for 4 hrs, and then evaporated in vacuo. Subsequent to the addition of water (100 mL), the resultant mixture was neutralized with 5% NaHCO₃ (50 mL), and then extracted with EtOAc. The organic layer was separated, washed with water, dried, and evaporated in vacuo. The obtained residue was eluted through a silica gel column with cyclohexane/EtOAc (4:1) to result in the title compound 4 (5.02 g, 16.3 mmol, yield 65%).

Detected Properties of the Title Compound:

IR (KBr) 3434, 1636, 1660 cm⁻¹.

¹H NMR ((CD₃)₂CO): δ 6.86 (1H, d, j=8.0 Hz, H-3′), 7.16 (1H, dd, J=8.0, 2.8 Hz, H-4′), 7.63 (1H, d, J=2.8 Hz, H-6′), 7.69 (1H, d, J=8.0 Hz, H-5), 7.85 (1H, d, J=16.0 Hz, H-α), 7.88 (1H, dd, J=8.0, 2.8 Hz, H-6), 8.06 (1H, d, J=16.0 Hz, H-β), 8.16 (1H, bs, OH-5′), 8.19 (1H, d, J=2.8 Hz, H-2), 12.16 (1H, s, OH-2′).

¹³C NMR ((CD₃)₂CO): δ 116.5 (C-6′), 120.2 (C-α), 121.3 (C-1′), 124.6 (C-3′), 126.9 (C-4′), 130.4 (C-6), 131.8 (C-2), 132.6 (C-5), 134.2 (C-1), 135.3 (C-3), 137.2 (C-4), 143.6 (C-β), 151.0 (C-5′), 158.6 (C-2′), 195.1 (CO).

EIMS (70 eV) m/z (% rel. int.): 308 [m]⁺ (8), 163 (21), 136 (100).

### Synthesis Example 5

### 3,4-Dichloro-2′,5′-diethoxychalcone (Compound 5)

A mixture of compound 4 (7.7 g, 25 mmol), ethyl iodide (8.1 g, 52 mmol) and potassium carbonate (15 g, 25 mmol) in DMF (50 mL) were stirred at room temperature for 18 hrs. The mixture was diluted with water and washed three times with water. The organic phase was dried over sodium sulfate, filtered, and concentrated in vacuo to give the product, which was purified via silica-gel column chromatography to result in the title compound 5 (5.3 g, 14.5 mmol, yield 58%).

Detected Properties of the Title Compound:

IR (KBr) 1657, 1596 cm⁻¹.

¹H NMR (CDCl₃): δ 1.40 (6H, m, 2×Me), 4.06 (4H, 2×OCH₂), 6.91 (1H, d, J=9.2 Hz, H-3′), 7.03 (1 H, dd, J=9.2, 3.2 Hz, H-4′), 7.24 (1H, d, J=3.2 Hz, H-6′), 7.40 (1H, dd, J=8.4, 2.0 Hz, H-6), 7.46 (1H, d, J=8.4 Hz, H-5), 7.51 (1H, d, J=16.0 Hz, H-α), 7.56 (1H, d, J=16.0 Hz, H-β), 7.66 (1H, d, J=2.0 Hz, H-2).

³C NMR (CDCl₃): δ 14.8 (Me), 15.0 (Me), 64.1 (OCH₂), 65.2 (OCH₂), 114.7 (C-6′), 115.1 (C-3′), 120.7 (C-α), 127.2 (C-4′), 128.7 (C-6), 129.2 (C-1′), 129.7 (C-2), 130.9 (C-5), 133.2 (C-1), 133.8 (C-3), 135.5 (C-4), 139.2 (C-β), 152.3 (C-5′), 153.1 (C-2′), 191.3 (CO).

EIMS (70 eV) m/z (% rel. int.): 366 [M+1]⁺ (25), 164 (100), 136 (79).

### Synthesis Example 6

### 3,4-Dichloro-2′-hydroxy-5′-propoxy-chalcone (Compound 6)

A mixture of compound 4 (7.7 g, 25 mmol), n-propyl iodide (8 84 g, 52 mmol), and potassium carbonate (15 g, 25 mmol) in DMF (50 mL) were treated according to the procedures as set forth in the above Synthesis Example 5 for compound 5 to result in the title compound 6 (4.04 g, 11.5 mmol, yield 46%).

Detected Properties of the Title Compound:

IR (KBr): 1646, 1577 cm⁻¹.

¹H NMR (CDCl₃): δ 1.07 (3H, t, Me), 1.83 (2H, m, CH₂), 3.94 (2H, t, OCH₂), 6.97 (1H, d, J=9.0 Hz, H-3′), 7.16 (1H, dd, J=9.0, 2.9 Hz, H-4′), 7.33 (1H, J=2.9 Hz, H-6′), 7.46 (1H, dd, J=8.3, 1.7 Hz, H-6), 7.54 (1H, d, J=16.0 Hz, H-α), 7.56 (1H, d, J=8.3 Hz, H-5), 7.74 (1H, d, J=1.7 Hz, H-2), 7.78 (1H, d, J=16.0 Hz, H-β), 12.21 (1H, s, OH).

¹³C NMR (CDCl₃): δ (10.6 (Me), 22.7 (CH₂), 70.7 (OCH₂), 114.0 (C-6′), 119.3 (C-α), 119.5 (C-1′), 121.8 (C-3′), 124.7 (C-4′), 127.7 (C-6), 129.9 (C-5), 131.0 (C-2), 133.4 (C-1), 134.6 (C-3, C-4), 142.5 (C-β), 151.3 (C-5′), 157.9 (C-2′), 192.8 (CO).

EMIS (70 eV) m/z (% rel. int.): 350 [M]⁺ (15), 178 (36), 136 (100).

### Synthesis Example 7

### 2′,5′-Dihydroxy-3-thienylchalcone (Compound 7)

Crude compound 4a (8.0 g, 25 mmol), 3-thiophenaldehyde (2.8 g, 25 mmol) and barium hydroxide octahydrate (4.29 g, 25 mmol) were reacted according to the procedures as set forth in the above Synthesis Example 4 for compound 4 to result in the title compound 7 (3.4 g, 13.8 mmol, yield 55%).

Detected Properties of the Title Compound:

IR (KBr): 3358, 1642 cm⁻¹.

¹H NMR (CDCl₃): 4.92 (1H, s, OH-5′), 6.93 (1H, d, J=8.8 Hz, H-3′), 7.06 (1H, dd, J=8.8, 3.2 Hz, H4′), 7.35 (1H, d, J=3.2 Hz, H-6′), 7.38 (1H, d, J=15.2 Hz, H-α), 7.39 (1H, dd, J=5.2, 2.8 Hz, H-4), 7.42 (1H, dd, J=5.2, 1.6 Hz, H-5), 7.65 (1H, dd, J=2.8, 1.6 Hz, H-2), 7.90 (1H, d, J=15.2 Hz, H-β), 12.41 (1H, s, OH-2′).

¹³C NMR (CDCl₃): δ 114.5 (C-6′), 119.4 (C-α), 119.7 (C-3′), 119.8 (C-1′), 124.8 (C-4′), 125.5 (C-5), 127.2 (C-2), 130.1 (C-4), 137.9 (C-3), 139.1 (C-β), 147.4 (C-5′), 157.7 (C-2), 193.5 (CO).

EMIS (70 eV) m/z (% rel. int.): 246 [M]⁺ (39), 136 (100).

### Synthesis Example 8

### 2′,2-Dithienylchalcone (Compound 8)

2-Acetylthiophene (2.8 g, 25 mmol), 2-thiophenaldehyde (2.8 g, 25 mmol) and barium hydroxide octahydrate (4.29 g, 25 mmol) were reacted according to the procedures as set forth in the above Synthesis Example 3 for compound 3 to result in the title compound 8 (3.86 g, 17.5 mmol, 70%).

Detected Properties of the Title Compound:

IR (KBr): 1638, 1571 cm⁻¹.

¹H NMR (CDCl₃): δ 7.08 (1H, dd, J=5.0, 3.6 Hz, H-4′), 7.17 (1H, dd, J=5.2, 4.0 Hz, H-4), 7.21 (1H, d, J=15.6 Hz, H-α), 7.36 (1H, d, j=5.2 Hz, H-5), 7.42 (1H, d, J=5.0 Hz, H-5′), 7.66 (1H, dd, J=3.6, 1.2 Hz, H-3′), 7.84 (1H, dd, J=4.0, 0.8 Hz, H-3), 7.96 (1H, d, J=15.6 Hz, H-β)

¹³C NMR (CDCl₃): δ 120.4 (C-α), 128.2 (C-4′), 128.3 (C-3′), 128.8 (C-5′), 131.6 (C-4), 132.1 (C-3), 133.8 (C-5), 136.4 (C-β), 140.1 (C-2), 145.5 (C-2′), 181.5 (CO).

EMIS (70 eV) m/z (% rel. int.): 220 [M]⁺ (100), 191 (68), 111 (57).

The Applicant synthesized compounds 1-8 in good yields. A comparison of the structures and analytical data of these compounds is summarized in the following Table 1.

TABLE 1
Structural comparison and analytical data of compounds 1-8

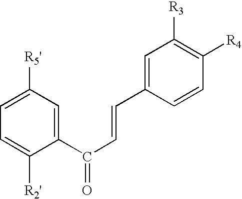

1-6

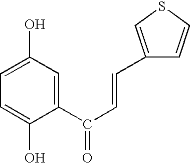

7

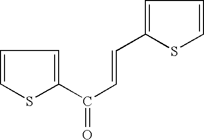

8

| No. | R₂′ | R₅′ | R₃ | R₄ | Formula^{a} | Mp (° C.) | Yield (%) |
|---|---|---|---|---|---|---|---|
| 1 | OMe | OMe | H | OH | C₁₇H₁₆O₄ | 127-8^{b} | 48 |
| 2 | OMe | OMe | H | Br | C₁₇H₁₅BrO₃ | 85-7^{b} | 45 |
| 3 | OMe | OMe | Cl | Cl | C₁₇H₁₄Cl₂O₃ | 121-3^{b} | 48 |
| 4 | OH | OH | Cl | Cl | C₁₅H₁₀Cl₂O₃ | 171-4^{b} | 65 |
| 5 | OEt | OEt | Cl | Cl | C₁₉H₁₈Cl₂O₃ | 152-3^{c} | 58 |
| 6 | OH | OPr | Cl | Cl | C₁₈H₁₆Cl₂O₃ | 97-9^{C} | 46 |
| 7 | | | | | C₁₃H₁₀O₃S | 156-7^{b} | 55 |
| 8 | | | | | C₁₁H₈OS₂ | 95-7 | 70 |

^{a}C and H analyses were within ± 0.4% of the theoretical values.
^{b}Crystallizing solvent: CHCl₃.
^{c}Crystallizing solvent: MeOH.

### Pharmacological Examples

In order to determine the biological activities of compounds 1-8 according to the present invention, the following pharmaceutical activity was performed, in which the anti-inflammatory activities of compounds 1-8 were studied in vitro for their inhibitory effects on chemical mediators released from mast cells, neutrophils, macrophages, and microglial cells. The experimental data were subjected to statistical analysis, and the data were presented as the means±s.e.m. Statistical analysis were performed using the Least Significant Difference Test methods after analysis of variance. P-values<0.05 were considered to be significant. Analysis of the regression line was used to calculate IC₅₀ values.

Concerning the reagents used in the following pharmacological examples, dextran and DMSO are respectively purchased from Pharmacia and Merck, and the rest may be available from Merck or Sigma Chem. Co., St. Louis, USA.

### Experiment 1

### Inhibitory Effect of Chalcone Derivative on Mast Cell Degranulation

Methods:

Heparinized Tyrode's solution was injected into the peritoneal cavity of exsanguinated rat (Sprague-Dawley, 250-300 g, 10-12 weeks old). After abdominal massage, cells in the peritoneal fluid were harvested and then separated in 38% bovine serum albumin (BSA) in glucose-free Tyrode's solution. Cell pellets were washed and suspended in Tyrode's solution with 0.1% BSA to 1×10⁶ cells/mL. Cell suspension was then preincubated at 37° C. with DMSO or a tested compound for 5 min. The final volume of DMSO in the reaction mixture was≦0.5%. Fifteen minutes after the addition of compound 48/80 (10 μg/mL), β-glucuronidase (phenolphthalein-β-glucuronide as substrate, 550 nm) and histamine (o-phthadialdehyde condensation, 350/450 nm) in the supernatant were determined (J. P. Wang et al. (1994), Eur. J. Pharmacol., 251, 35-42). After treatment of the cell suspension with Triton X-100, the total content of β-glucuronidase and histamine was measured, and the percentage release was calculated. In the experiment, Mepacrine was used as a positive control.

Results:

It can be seen from Table 2 that compound 4 caused a concentration-dependent inhibition of mast cell degranulation stimulated with compound 48/80 (10 μg/mL) The results shown in Table 2 indicate that etherifying the 2′,5′-diphenolic or substituting the 2′,5′-dihydroxyphenyl or substituting the B ring of 2′,5′-dihydroxychalcone did not enhance the inhibitory effects on mast cells degranulation, while 2′,5′-dihydroxychalcone with a 3,4-dichlorinated B ring significantly enhanced the inhibitory effects.

TABLE 2
Inhibitory effects of chalcone derivatives on the release of
β-glucuronidase and histamine from rat peritoneal mast cells
stimulated with compound 48/80

IG₅₀ (μM)^{a}

| Compound | β-Glucuronidase | Histamine |
|---|---|---|
| 1 | >30 (8.3 ± 2.3) | >30 (10.0 ± 5.1) |
| 2 | >30 (28.6 ± 3.4) | >30 (21.7 ± 1.5) |
| 3 | >30 (44.3 ± 2.3) | >30 (35.2 ± 1.0) |
| 4 | 8.9 ± 1.6 | 11.2 ± 1.8 |
| 5 | >30 (5.5 ± 4.7) | >30 (12.9 ± 7.9) |
| 6 | >30 (49.2 ± 2.0) | >30 (47.6 ± 2.4) |
| 7 | >30 (49.1 ± 13.2) | >30 (34.0 ± 11.0) |
| 8 | >30 (17.3 ± 2.6) | >30 (20.9 ± 1.2) |
| Mepacrine | 32.2 ± 3.6 | 48.5 ± 3.8 |

^{a}When 50% inhibition could not be reached at the highest concentration, the % of inhibition is given in parentheses. Data are presented as means ± s.e.m. (n = 3-5). Mepacrine was used as a positive control.

### Experiment 2

### Inhibitory Effect of Chalcone Derivative on Neutrophil Degranulation

Methods:

Blood was withdrawn from rat (Sprague-Dawley, 250-300 g, 10-12 weeks old) and mixed with EDTA. After dextran sedimentation, Ficoll-Hypaque separation and hypotonic lysis of the residual erythrocytes, neutrophils were washed and suspended in Hanks' balanced salt solution (HBSS) to 1×10⁷ cell/mL (J. P. Wang et al (1995), Eur. J. Pharmacol., 288, 341). Cell suspension was preincubated at 37° C. with DMSO or drugs for 10 min, and then challenged with fMLP (1 μM)/CB (5 μg/mL). The final volume of DMSO in the reaction mixture was≦0.5%. Forty-five minute later, the lysozyme (Micrococcus lysodeikticus as substrate, 450 nm) (D. R. Absolom (1986), Methods Enzymol., 132, 92) and β-glucuronidase in the supernatant were determined. After treatment of the cell suspension with Triton X-100, the total content of lysozyme and β-glucuronidase was measured and the percentage release was calculated. In the experiment, Trifluoperazine was used as a positive control.

Results:

FMLP/CB stimulated the release of β-glucuronidase and lysozyme from rat neutrophils. Referring to Table 3, compounds 1-4, 6 and 7 were shown to have potent and concentration-dependent inhibitory effects on neutrophil degranulation.

The results shown in Table 3 clearly indicate that the increase in lipophilicity of compound 4 or substituting the 3,4-dichlorinated B ring of compound 4 did not enhance the inhibitory effect. Compounds 5 and 8 had no significant inhibitory effects in this respect. Hence the enone moiety of chalcones further appears to be required for the inhibition of neutrophils degranulation (H. K. Hsieh et al. (2000), J. Pharm. Pharmacol., 52, 163-171).

TABLE 3
Inhibitory effects of chalcone derivatives on the release of
β-glucuronidase and lysozyme from rat neutrophils stimulated
with fMLP/CB

IC₅₀ (μM)^{a}

| Compound | β-Glucuronidase | Lysozyme |
|---|---|---|
| 1 | 20.6 ± 2.0 | 28.3 ± 1.9 |
| 2 | 5.6 ± 0.3 | 5.7 ± 0.3 |
| 3 | 5.0 ± 0.3 | 5.1 ± 0.1 |
| 4 | 1.3 ± 0.1 | 1.2 ± 0.1 |
| 5 | >30 (30.6 ± 4.1) | >30 (27.1 ± 5.0) |
| 6 | 6.2 ± 0.3 | 6.6 ± 0.4 |
| 7 | 4.6 ± 0.4 | 6.7 ± 0.3 |
| 8 | >30 (46.6 ± 0.7) | >30 (30.0 ± 1.4) |
| Trifluoperazine | 18.9 ± 2.1 | 18.3 ± 0.9 |

^{a}When 50% inhibition could not be reached at the highest concentration, the % of inhibition is given in parentheses. Data are presented as means ± s.e.m. (n = 3-5). Trifluoperazine was used as a positive control.

### Experiment 3

### Inhibitory Effect of Chalcone Derivative on Superoxide Anion Generation in Neutrophil

Methods:

Superoxide anion generation was measured in terms of superoxide dismutase-inhibitable cytochrome c reduction (M. Market et al. (1984), Methods Enzymol., 105, 358). Neutrophil suspension was preincubated with DMSO or a tested compound for 3 min, and then superoxide dismutase or Hank's balanced salt solution (HBSS) was added into the blank and test wells, respectively. The final volume of DMSO in the reaction mixture was≦0.5%. After addition of cytochrome c, reaction was initiated by challenge with formyl-Met-Leu-Phe (fMLP) (0.3 μM)/cytochalasin B (CB) (5 μg/mL) or phorbol myristate acetate (PMA) (3 nM). Thirty minutes later, the reaction was terminated by centrifugation and the absorbance changes of supernatant were monitored at 550 nm in a microplate reader. In the experiment, Trifluoperazine was used as a positive control

Results:

FMLP/CB or phorbol myristate acetate (PMA) (3 nM) stimulated superoxide anion generation in rat neutrophils. Referring to Table 4, compounds 1-4, 6, and 8 are shown to have potent inhibitory effects on fMLP/CB-induced superoxide anion generation, while compounds 5 and 7 appear not to have significant inhibitory effects. These results indicate that the introduction of a lipophilic alkyl group at the C-2′ and C-5′ positions of compound 4 might attenuate its inhibitory effects on fMLP/CB-induced responses. In addition, the essential role of the enone moiety of chalcones in the inhibition of fMLP/CB-stimulated superoxide anion generation reconciled with the earlier observation (H. K. Hsieh et al. (2000), J. Pharm. Pharmacol., 52, 163-171). Compound 4 was more potent than the positive control, trifluoperazine.

It has been reported that fMLP and PMA activate NADPH oxidase to produce superoxide anion through different cellular signaling mechanisms (A. W. Segal and A. Abo (1993), Trends Biochem. Sci., 18, 43-47). The observed results that compounds 1-8 have no appreciable effect on PMA-induced response suggest the involvement of PMA-independent signaling pathway.

TABLE 4
Inhibitory effects of chalcone derivatives on superoxide anion
generation in rat neutrophils stimulated with fMLP/CB or PMA

IC₅₀ (μM)^{a}

| Compound | fMLP/CB | PMA |
|---|---|---|
| 1 | 19.5 ± 3.5 | >30 (36.3 ± 6.5) |
| 2 | 8.0 ± 2.4 | >30 (22.9 ± 6.9) |
| 3 | 12.9 ± 0.4 | >30 (−27.1 ± 9.2) |
| 4 | 0.6 ± 0.2 | >10 (21.9 ± 8.7) |
| 5 | >30 (32.4 ± 12.2) | >30 (13.2 ± 3.7) |
| 6 | 17.7 ± 7.2 | >30 (49.9 ± 3.4) |
| 7 | >1 (43.4 ± 6.1) | >1 (23.5 ± 10.4) |
| 8 | 24.3 ± 3.2 | >30 (35.6 ± 1.6) |
| Trifluoperazine | 12.9 ± 1.0 | 9.7 ± 1.1 |

^{a}When 50% inhibition could not be reached at the highest concentration, the % of inhibition is given in parentheses. Data are presented as means ± s.e.m. (n = 3-5). Trifluoperazine was used as a positive control.

### Experiment 4

### Inhibitory Effect of Chalcone Derivative on NO Production in Macrophage and Microglial Cell

Methods:

A. Culture and Drug Treatment of Macrophage:

RAW 264.7 mouse macrophage-like cell line (American Type Culture Collection (ATCC), PO Box 1549, Manassas, Va. 20108 USA) was plated in 96 well tissue-culture plates in Dulbecco's Modified Eagle Medium (DMEM) supplemented with 10% fetal calf serum (FCS), 100 units/ml of penicillin and streptomycin at 2×10⁵ cells/200 μL per well. Cells were allowed to adhere overnight. The cells were treated with DMSO or a tested compound at 37° C. for 1 hr. followed by stimulation with 1 μg/mL of LPS (Escherichia coli, serotype 0111:B4) for 24 hrs. Thereafter, the medium in each well was collected and stored at −70° C. until use. The final volume of DMSO in the reaction mixture was≦0.5%.

B. Culture and Drug Treatment of Microglial Cell:

Cells of murine microglial cell line N9 (kindly provided by Dr. P. Ricciardi-Castagnoli, CNR Cellular and Molecular Pharmacology Center, Italy) (S. B. Corradin et al. (1993), Glia, 7, 255-262) were suspended in Iscove's Modified Dulbecco's medium containing 5% heat-inactivated FCS and antibiotics and then plated into 96-well tissue-culture plates at 8×10⁴ cells/200 μL per well. The cells were treated with DMSO or a tested compound at 37° C. for 1 hr, followed by stimulation with LPS (10 ng/mL)/IFN-γ (10 unit/mL) for 24 hrs. Thereafter, the medium in each well was collected and stored at −70° C. until use. The final volume of DMSO in the reaction mixture was<0.5%

C. NO Determination:

The production of NO in cell medium was determined by measuring the content of nitrite based on the Griess reaction (L. Mingghetti et at (1997), Glia, 19, 152-160). Briefly, 40 μL of 5 mM sulfanilamide, 10 μL of 2 M HCl, and 20 μL of 40 mM naphthylethylenediamine were added into 150 μL culture medium in sequence. After 10 min of incubation at room temperature, absorbance was measured at 550 nm in a microplate reader. A standard nitrite curve was generated in the same fashion, using NaNO₂.

Results:

Treatment of RAW 264.7 macrophage-like cells with LPS (1 μg/mL) for 24 hrs induced NO production as assessed by measuring the accumulation of nitrite, a stable metabolite of NO, in the media based on Griess reaction. As shown in Table 5, LPS induced a significant increase of NO production and this effect was suppressed by compounds 1 and 5 in a concentration-dependent manner.

The parallel inhibition of NO production in N9 microglial cells as well as in RAW 264.7 cells by compounds 1-8 was also observed. The chalcones with increased lipophilicity are shown to have significantly enhanced inhibitory effects on NO production. The chalcones derivatives of this invention may be potential leading compounds for the development of more potent drugs to inhibition of NO production in macrophages.

TABLE 5
Inhibitory effects of chalcone derivatives on the accumulation of
NO₂⁻ in the culture media of RAW 264.7 cells
in response to LPS and N9 cells in response to LPS/IFN-γ

IC₅₀ (μM)^{a}

| Compound | RAW 264.7 cells | N9 cells |
|---|---|---|
| 1 | 14.6 ± 0.1 | 17.8 ± 0.2 |
| 2 | >10 (8.9 ± 4.0) | >10 (−5.6 ± 1.1) |
| 3 | >3 (2.0 ± 3.1) | >3 (4.3 ± 3.1) |
| 4 | >1 (−4.9 ± 1.9) | >1 (−10.3 ± 1.5) |
| 5 | 4.9 ± 0.3 | 3.7 ± 0.1 |
| 6 | >10 (7.5 ± 0.4) | >30 (26.4 ± 2.3) |
| 7 | >3 (9.7 ± 2.9) | >3 (10.6 ± 8.2) |
| 8 | >30 (12.5 ± 2.2) | >30 (15.2 ± 0.5) |
| L-NAME^{b} | 0.51 ± 0.02 mM | 0.63 ± 0.01 mM |

^{a}When 50% inhibition could not be reached at the highest concentration, the % of inhibition is given in parentheses. Data are presented as means ± s.e.m. (n = 3-5).
^{b}N^{ω}-nitro-L-arginine methyl ether (L-NAME) was used as a positive control.

The above experiments demonstrate that the chalcone derivatives of this invention exert potent inhibitory effects on the release of chemical mediators from inflammatory cells. NO plays a central role in macrophage-induced cytotoxicity and has been demonstrated to implicate in the pathology of central neurologic diseases and also in the peripheral tissue damage associated with acute and chronic inflammation, (L. Bo et al. (1994), Ann. Neurol., 36, 778; Laskin, D.-L.; K. J. Pendino (1995), Annu. Rev. Pharmacol. Toxicol., 25, 655) and septic shock (Thiermermann and J. R. Vane (1990), Eur. J. Pharmacol., 182, 591-595). The inhibition of NO production by compounds 1 and 5 in macrophages and 5 in microglial cells may have value in the therapeutic treatment or prevention of certain central as well as peripheral inflammatory diseases associated with the increase of NO production.

All patents and literature references cited in the present specification are hereby incorporated by reference in their entirety. In case of conflict, the present description, including definitions, will prevail.

While the invention has been described with reference to the above specific embodiments, it is apparent that numerous modifications and variations can be made without departing from the scope and spirit of this invention. It is therefore intended that this invention be limited only as indicated by the appended claims.

We claim:

1. A compound of formula (I): 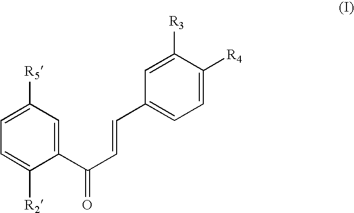
or a pharmaceutically acceptable salt or solvate thereof,
wherein
R₂′ and R₅′ are the same and represent OR, where R is H or a C₁₋₆ alkyl group; or R₂′ is OH and R₅′ is propoxy;
R₃ represents H or Cl; and
R₄ represents OH, Cl or Br;
with the proviso that when R₃ is H, R₄ cannot be Cl.

2. The compound of claim 1, wherein both R₃ and R₄ are Cl.

3. The compound of claim 2, wherein both R₂′ and R₅′ are OH.

4. The compound of claim 2, wherein both R₂′ and R₅′ are ethoxy.

5. The compound of claim 2, wherein R₂′ is OH and R₅′ is propoxy.

6. The compound of claim 1, wherein both R₂′ and R₅′ are methoxy.

7. The compound of claim 6, wherein R₃ is H and R₄ is OH.

8. The compound of claim 6, wherein R₃ is H and R₄ is Br.

9. The compound of claim 6, wherein both R₃ and R₄ are Cl.

10. A pharmaceutical composition comprising a compound of formula (I) as claimed in claim 1, or a pharmaceutically acceptable salt or solvate thereof, and, optionally, a pharmaceutically acceptable carrier.

11. A method for treating an inflammatory disorder in a subject, comprising administering to a subject in need of such treatment a therapeutically effective amount of a compound as claimed in claim 1, or a pharmaceutically acceptable salt or solvate thereof.

12. The method of claim 11, wherein the inflammatory disorder is associated with the release of chemical mediators from inflammatory cells selected from macrophages, neutrophils and microglial cells.

13. The method of claim 11, wherein the inflammatory disorder is selected from pathologies of central neurologic diseases, peripheral tissue damages associated with acute or chronic inflammation, inflammatory/neuronal conditions observed in aging and Alzheimer's disease, and septic chock.

14. The method of claim 11, wherein the inflammatory disorder is associated with NO production by inflammatory cells selected from macrophages, neutrophils and microglial cells.

15. A compound of formula (II): 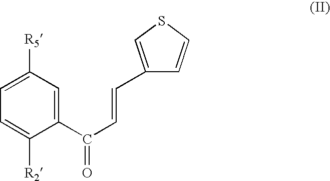
or a pharmaceutically acceptable salt thereof,
wherein both R₂′ and R₅′ are OH.

16. A pharmaceutical composition comprising a compound of formula (II) as claimed in claim 15, or a pharmaceutically acceptable salt or solvate thereof and, optionally, a pharmaceutically acceptable carrier.

17. A method for treating an inflammatory disorder in a subject, comprising administering to a subject in need of such treatment a therapeutically effective amount of a compound as claimed in claim 15, or a pharmaceutically acceptable salt or solvate thereof.

18. The method of claim 17, wherein the inflammatory disorder is associated with the release of chemical mediators from inflammatory cells selected from macrophages, neutrophils and microglial cells.

19. The method of claim 17, wherein the inflammatory disorder is selected from pathologies of central neurologic diseases, peripheral tissue damages associated with acute or chronic inflammation, inflammatory/neuronal conditions observed in aging and Alzheimer's disease, and septic chock.

20. The method of claim 17, wherein the inflammatory disorder is associated with NO production by inflammatory cells selected from macrophages, neutrophils and microglial cells.

21. A compound of formula (III): 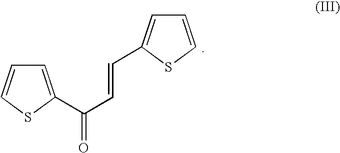
or a pharmaceutically acceptable salt thereof.

22. A pharmaceutical composition comprising a compound of formula (III) as claimed in claim 21, or a pharmaceutically acceptable salt or solvate thereof, and, optionally, a pharmaceutically acceptable carrier.

23. A method for treating an inflammatory disorder in a subject, comprising administering to a subject in need of such treatment a therapeutically effective amount of a compound as claimed, in claim 21, or a pharmaceutically acceptable salt or solvate thereof.

24. The method of claim 23, wherein the inflammatory disorder is associated with the release of chemical mediators from inflammatory cells selected from macrophages, neutrophils and microglial cells.

25. The method of claim 23, wherein the inflammatory disorder is selected from pathologies of central neurologic diseases, peripheral tissue damages associated with acute or chronic inflammation, inflammatory/neuronal conditions observed in aging and Alzheimer's disease, and septic chock.

26. The method of claim 23, wherein the inflammatory disorder is associated with NO production by inflammatory cells selected from macrophages, neutrophils and microglial cells.
